(19) United States
(12) Patent Application Publication
(10) Pub. No.: US 2012/0034596 A1
(43) Pub. Date: Feb. 9, 2012

(54) DEVICE FOR AUTOMATICALLY CULTIVATING CELLS IN PARALLEL

(75) Inventors: Josef Seidl, Arnstorf (DE); Michael Wiechmann, Haar (DE)

(73) Assignee: PAN-SYSTECH GMBH, Aidenbach (DE)

(21) Appl. No.: 13/257,148

(22) PCT Filed: Apr. 22, 2010
(86) PCT No.: PCT/DE2010/000454

§ 371 Date: Sep. 16, 2011

(30) Foreign Application Priority Data
Apr. 22, 2009 (DE) 10 2009 018 325.6
Apr. 22, 2009 (DE) 20 2009 005 898.0

(51) Int. Cl. C12Q 3/00; C12M 1/36; C12M 1/38; C12M 1/00

(52) U.S. Cl. 435/3; 435/289.1; 435/286.5

(57) ABSTRACT

A device for automatically cultivating cells in parallel in cell culture vessels, in particular microtiter (MT) plates (2), having a housing (23) in which are disposed an observation unit (3) comprising at least one microscope (7) and at least one camera (6), a receptacle device (4) for receiving the cell culture vessels, and a fluid distribution unit (5) for automatically filling and/or emptying the cell culture vessels, in particular the wells (17) of microtiter (MT) plates (2), with fluid, wherein the climatic conditions in the device, in particular the gas composition and temperature, can be controlled in a closed loop at least in the region of the cell culture vessels, wherein a climate chamber (21) is provided in the housing, in which a desired temperature and/or a particular gas composition can be adjusted automatically, and in which the receptacle device (4); having the cell culture vessels (2) is at least partially integrated.

The present invention relates to a device for automatically cultivating cells in parallel in cell culture vessels, in particular microtiter (MT) plates.

Cultivating isolated cells in vitro is recognized as a valuable tool in biomedical research to secure reproducible data. Cell cultivation, however, is still mainly implemented manually, even today. Since significant variations in manual processing are unavoidable, often accompanied by lack of proper sterility, reproducibility and cell quality—which are directly responsible for a successful application, e.g. in developing active substances—are diminished. In addition to this, the human factor always constitutes an added risk of wrong or unstable handling, especially also when working with a large number of different cell lines. Apart from the quality, thruput in manual cell cultivation is limited or scalable only by a corresponding team of coworkers.

Cell cultures are often used in the pharmaceuticals, cosmetics and biotech industry, among others, in the search for new active mechanisms and substances in pharmaceuticals and pesticides having since become indispensable. Only standardized, automated cell culture methods with a reliable evaluation of all cell culture parameters permit a long-term comparison of the substances and their active mechanisms in thus constituting, among other things, an essential base for the research and development activity in the biopharmaceuticals industry in the fields of cell culture, media development and the development of active substances.

Because of their high manual complexity due to extensive serial testings, cell culture methods known hitherto from prior art, prove to be difficult to document and reproduce. Currently, because of the lack of flexibility and automation in cell culture, each and every wanted change in the culture conditions results in high additional expense for lack of flexibility and automation of the cell culture, necessitating costly human resources, adding to the costs for the user.

The present invention is based on the object of providing a device for automatically cultivating cells in parallel in cell culture vessels, in particular microtiter (MT) plates which overcomes the drawbacks of prior art.

This object is achieved by a device having the features as it reads from claim 1.

Thanks to the observation unit including a microscope and a camera, continuous observation and optical evaluation of cell growth and cell morphology are now possible.

Cell culture vessels may be e.g. cell culture flasks, preferably, however, they are MT plates.

The MT plates are automatically filled by the fluid distribution unit. Any fluid not required or stale is emptied or replenished respectively.

The modules of the system (fluid distribution unit, receptacle device, climate chamber and fluid distribution unity are arranged in a special housing so that the device functions as a so-called sterile bench permitting open fluidics and media supply since the housing greatly minimizes the risk of bacterial contamination.

The complete device functions as a rule automatically, i.e. the main operating steps in cultivating the cells, such as e.g. filling and emptying the wells of the MT plates, microscopic observation and evaluation, are all programmed to occur automatically, so that all steps can be implemented in the closed system of the sterile bench with no need to remove, for example, MT plates from the system. Since the climatic conditions in the device are controlled in a closed loop the climate (temperature and/or gas mixture) can be adapted from without to the requirements of the cell cultures in each case.

The device in accordance with the invention comprises a climate chamber in which a wanted climate, namely a wanted temperature and/or a wanted humidity or a specific gas composition can be set, preferably automatically, and in which the receptacle device with the cell culture vessels, particularly the MT plates, is integrated at least in part, the climate chamber being arranged in the housing. It is the climate chamber that makes it possible to generate a specific climate only within a limited range within the housing. As a rule all cell culture vessels, particularly the MT plates including the cell cultures sited therein are arranged within the climate chamber in which a specific climate optimized for the cell cultures concerned is then set. The temperature is preferably set to values ranging from 20 to 45° C. As a rule the CO₂ concentration is set in the range from 0 to 20%, whilst the oxygen concentration within the climate chamber ranges as a rule from 10 to 40%. Sensing the climate within the climate chamber is preferably done by corresponding sensors within the climate chamber. In addition, the climate chamber achieves a further wailing-off of the cell cultures from the ambience in thus further minimizing the risk of the cell cultures being bacterially contaminated in the cell culture vessels.

In one particularly preferred embodiment of the device in accordance with the invention separate gas vessels are provided containing diverse gases, preferably O₂, CO₂ and N₂, each gas streaming from the gas flasks being controllable and wherein specific quantities of each gas are directed into a mixing chamber from where the resulting gas mixture is forwarded into the climate chamber. It is by these ways and means that a precisely predefined gas mixture is produced which is first assayed as to the wanted composition before being forwarded from the mixing chamber into the climate chamber. Setting a specific gas composition is particularly important in cultivating the cells. Whilst, on the one hand, tumor cells, for instance, grow in much less oxygen than is usual in the ambience, on the other, damaged neuronal cells, for example, are to be evaluated at much higher O₂ concentrations than those of the sited tumor cells. To advantage the gas mixture produced in the mixing chamber is brought to a specific temperature before the gas mixture is directed into the climate chamber, it being by these ways and means that optimum conditions for the various cells to be cultivated are achieved.

By means of the receptacle device MT plates varying, for example, in number and number of wells (e.g. 6, 12, 24, 48, 96, 384 wells per MTP) are received. In one preferred embodiment of the device in accordance with the invention the cell culture vessels, particularly the MT plates, are transportable preferably in the region of the observation unit and/or in the region of the fluid distribution unit, preferably automatically by means of a carriage or robotic arm to/from the climate chamber. It is by these ways and means that specific, selected cell culture vessels, particularly MT plates, can be shortly transported from the climate chamber for filling with fluid. Specific cell culture vessels can also be transported into the region of the observation unit for observation. Shortly transporting the cell culture vessels from the climate chamber has no adverse affect on the climate within the climate chamber. Automatically transporting the cell culture vessels to/from the climate chamber does away with having to manually move the MT plates within the system with the high risk of contamination.

Preferably the observation unit and/or the fluid distribution unit are arranged outside of the climate chamber with the advantage, among other things, that these units are not exposed to the sometimes extreme changes in climate with the climate chamber by, for instance, protecting them from high humidity which could be detrimental to the units whilst, in addition, preventing contamination of the cell cultures by these units.

To advantage the receptacle device comprises at least two receptacle units arranged preferably stacked and preferably including means for supporting the cell culture vessels, particularly MT plates. Stacking the receptacle units makes for great space saving, it also contributing towards reducing system costs whilst enabling substantially more cell culture vessels to be accommodated in the climate chamber. These supporting means may be configured, for example, as recesses within the receptacle units shaped to nest the cell culture vessels, particularly MT plates.

In another embodiment of the device in accordance with the invention a cell culture vessel, particularly MT plate is devised moveable, preferably controlled, in the region of the observation unit.

To advantage the optics (microscope incl. camera) of the observation unit and/or the cell culture vessel for observation are moveable, the movements of the camera or cell culture vessels preferably being controllable.

The microscope used in the optics of the device in accordance with the invention may be a normal (transmitted light) microscope or also a phase contrast microscope or, indeed, any other detection device for observing and/or analyzing cells. Since, however, fluorescent microscopy can also be done with the device in accordance with the invention it is often an advantage when the microscope is a fluorescent microscope.

The optics of the device in accordance with the invention preferably feature AUTO focussing. As a rule the optics furthermore feature AUTO changing of the objective lens.

The observation unit is preferably engineered for online evaluating the morphology, physiology, response and growth of cells, evaluating the cell culture data being done as a rule in a PC with specifically devised software and user surface.

In another preferred embodiment of the device in accordance with the invention the fluid distribution unit includes a plurality of receptacles for containing diverse fluids such as e.g. cell solutions, nutrients and stains as well as a microdispenser for dispensing the fluids defined and controlled into the cell culture vessels, particularly into the MT plate wells and preferably also for removing the fluids from the cell culture vessels, particularly from the MT plate wells, the movements and actions of the microdispenser preferably being controllable. By configuring the fluid distribution unit with a plurality of receptacles for siting diverse fluids, a plurality thereof can be added, for example, to specific wells of MT plates. When, for instance, the operation requires fluorescent microscopy, in some wells stains in addition to cell solutions and nutrients can be introduced for the fluorescent microscopy, whereas, when some wells need to be freed of fluids, this can likewise be done by the microdispensing unit of the fluid distribution unit. The receptacles may be positioned in a circle around the microdispensing unit, in which case the microdispensing unit is preferably engineered to rotate and/or move up and down to enable the microdispensing unit to locate the individual receptacles.

To advantage the cell culture vessels, particularly the MT plates and preferably also the wells thereof are indexed by each plate and preferably each well as a rule being assigned an index which is recognized by the system, particularly by the software as described below.

For particularly accurate control of the individual steps in processing and for a precise assignment of the cell culture substances (for example, cells, fluids) to the individual culture vessels and assignment of the latter to the individual processing stations (fluid station, incubation station, optics station) indexing is preferably scheduled in 4 phases, In the first indexing phase each culture vessel is indexed for explicit assignment and identification by being provided with an internal index/number (for example MTP 1 to NTP 24). In the second indexing phase each sub-vessel of a culture vessel (as a rule each well of an MT plate) is indexed. In the third indexing phase each location in the receptacle device (particularly in the climate chamber) is indexed for explicit assignment and identification (for example MTP 1 to MTP 24), In the fourth indexing phase each process site of the system as a whole is indexed for explicit assignment, identification and control (for example fluid station=1, receptacle station=2, optics station=3).

It is particularly preferred that the cell culture vessels are configured transparent to permit application of all kinds of microscopy, including particularly fluorescent microscopy. The MT plates used are preferably dimensioned very thin so that the objective lenses of the microscope can be positioned as close as possible to a cell culture for observation. The MT plates bottomed for example by a thin film may be made of any transparent material such as e.g. glass, plastics, etc.

As already explained above as an alternative to MT plates any other cell culture vessels, for instance cell culture flasks such as T25 or T75 or also closed AUTO flasks may be used. In one preferred embodiment the aforementioned diverse culture vessels may be used both alternatively and also combined, for which purpose, for example, the receptacle device for receiving the cell culture vessels comprises diverse adapters to adapt it to the differing shapes and sizes of culture vessels.

In another particularly preferred embodiment of the device in accordance with the invention sensors for sensing all relevant parameters, particularly temperature, CO₂concentration, O₂ concentration, N₂ concentration, humidity and/or the supply of fluids into the cell culture vessels, particularly into the wells of the MT plates are featured as the basis for fully automated sensing and control of the climatic conditions and fluid supply to the cell culture vessels.

In another particularly preferred embodiment the device in accordance with the invention is characterized by a computerized sensing and control system for automatically sensing and controlling at least one, preferably all of the following parameters or steps in processing
a) temperature, particularly in the climate chamber;
b) O₂ concentration in the gas, particularly in the climate chamber;
c) CO₂ concentration in the gas, particularly in the climate chamber;
d) N₂ concentration in the gas, particularly in the climate chamber;
e) filling and/or emptying selected cell culture vessels, particularly selected wells of selected MT plates with selected quantities of selected fluids at selected times;
f) microscopic observation and evaluation of cell cultures in selected cell culture vessels particularly in selected wells of selected MT plates at selected times by a selected technique, such as e.g. fluorescence, transmitted light or phase contrast microscopy.

Preferably all parameters or steps in operating the system are saved, as of which, for example, the climatic conditions are automatically controlled in a closed loop by the system. Should one or more parameters deviate from these saved values in the course of cell cultivation cycles (e.g. due to briefly opening the climate chamber for transporting an MT plate thereinto or therefrom, this is automatically controlled in a closed loop as a correction to the saved value. For specifically filling selected wells of selected MT plates with selected quantities of selected fluids the MT plates and preferably also the wells thereof are indexed to thus greatly facilitate entering them into the system for later ready recognition thereof by the system. With the aid of the optics the cell cultures are preferably continuously imaged and saved in the system. The system also preferably recognizes certain changes to the cell cultures in responding to such changes by correcting the closed loop control of the aforementioned parameters or steps in operation.

One preferred embodiment of the device in accordance with the invention is characterized by a capper/uncapper for capping and uncapping cell culture vessels preferably automatically (see also description of the FIGS.).

In another preferred embodiment of the device in accordance with the invention a controlled selective use of pipettes for disposable or multiple application in the fluid distribution unit is possible in preventing cross-contamination between individual culture vessels and wells. In the device in accordance with the invention two types of pipettes are used, the first being so-called working pipettes intended for multiple application during cultivation, necessitating the pipettes being received sterile and returned sterile by being parked in sterile boxes within the device in accordance with the invention. In the region of the fluid distribution unit a robotic arm as a rule retrieves the working pipettes sterile from the reception boxes, after which the wanted fluid actions are implemented at the cell culture vessels, the robotic arm of the fluid distribution unit then returning the working pipettes sterile in the reception box. This working cycle can be repeated multiply during cell cultivation.

In addition to the working pipettes, disposable pipettes find application as a rule, used only once during cell cultivation, making it necessary to provide disposable sterile reception and safe disposal after implementation of the disposable action. The disposable pipettes are housed in sterile disposable boxes in the region of of the fluid station. In operation the fluid robotic arm picks a disposable pipette from a receptacle box sterile, after which the wanted fluid action is implemented at a cell culture vessel, particularly a MT plate. Following the action, the robotic arm ejects the disposable pipette into a waste box. This work cycle takes place once only during a cell culture with a disposable pipette.

The present invention relates furthermore to a method for automatically cultivating cells in parallel in cell culture vessels, in particular microtiter (MT) plates located in a cell culture system, particularly in a device as set forth in any of the claims 1 to 15, comprising the following steps in operation:
a) filling the system with cell culture vessels;
b) filling the system with receptacles containing the fluids to be used such as cell culture medium, cell suspension, test substance, stain;
c) componenting the system with receptacles containing gases such as O₂, CO₂, N₂ to be used;
d) programming the system for automatically setting at least one of the following parameters:
   temperature
   O₂ concentration in the gas
   CO₂ concentration in the gas
   N₂ concentration in the gas
e) programming the system for automatically filling and/or emptying cell culture vessels, particularly selected wells of selected MT plates with selected quantities of selected fluids at selected times,
f) programming the system for microscopic observation and evaluation of cell cultures in selected cell culture vessels particularly in selected wells of selected MT plates at selected times by a selected technique, such as e.g. fluorescence, transmitted light or phase contrast microscopy or any other method of detection for observing and/or analysing cells

In the device or method in accordance with the invention the system automatically controls the climatic conditions in the sense of a closed circuit controller to maintain the climatic conditions constant. Furthermore the system implements microscopic evaluation as programmed for the microscopy.

For optical evaluation a plurality of freely selectable points of interest (POI) can be defined in each culture vessel or in sub-units (e.g. POI 1 to 10 in well 2 of culture vessel 4), a specific control software being freely selected at the units as to which POI in which culture vessel are to be evaluated at which point in time and by which optical method (for example, fluorescence, transmitted light or phase contrast microscopy). It is particularly preferred that each POI can be travelled to by the travel unit (cross table) in three axes (both horizontally and vertically) for focussing, imaging, evaluation and memorizing by the system. The ability of imaging and memorizing POIs three-dimensionally permits focussing in various planes in a culture vessel respectively in a sub-unit thereof.

Each and every POI can be travelled to and evaluated with high precision of better than 1 micron, it being possible, however, in addition to single takes and evaluations to also achieve speeded-up takes and dynamic evaluation of each and every POI by a combination of the single images. Possible furthermore, as a rule, is automatic analysis of the optical image information (for instance, number, movement and shape of cells, stain information, fluorescent microscope cell components, number of stained cells) by means of specific software and units.

Furthermore, stale medium and fresh medium can be removed and added respectively from/to the system.

Filling the system with cell culture vessels and filling the system with receptacles containing the fluids to be used is done as a rule manually, this also applying to componenting the system with receptacles containing the gases to be used, for which purpose the device in accordance with the invention may comprise a further housing containing the cited gas flasks.

In summary, among other things, the device and method in accordance with the invention now features the following advantages:
automatically cultivating cells in parallel in cell culture vessels, particularly MT plates in an overall system with automatic sensing of all cell culture parameters (e.g. temperature, CO₂ concentration, O₂ concentration, N₂ concentration, humidity, fluid support, active substances);
low costs;
open system platform solution for the biopharmaceuticals industry achieving the development of both new, improved cell lines as well as efficient screening and development of new active substances with high reproducibility;
compact and uniform, intuitive operation requiring simple to achieve operating personnel skills;
good economy of use due to minimum variation in the use of more-expensive components;
engineered simple, ergonomic and modular with long-life components featuring high biocompatability;
simultaneous cultivation of diverse cells in parallel under identical conditions;
continuous microscopic observation, documentation and memorizing of all relevant data for facilitated retrieval and evaluation;
direct measurement of effects due to a change in the culture conditions with possibility for evaluation depending on the media composition;
instant recognition of morphological changes with improvment of development quality due to highly informative results;
setting optimum concentrations of individual substances and smooth change in the culture conditions now possible;
time needed for development now shortened by a factor 2 to 4 due to parallelization (reducing e.g. the time needed to optimize media from hitherto 12 months to 3 to 6 months);
costs of development now reduced by a factor 2 to 3 due to automation (reducing e.g. the costs needed to optimize media from hitherto 75,000 EUR to 25,000 to 40,000 EUR)
automated implementation of complex cell cultures e.g. for assaying toxic effects reducing the need for and number of tests on animals.

The fields of application open to the device and method in accordance with the invention are (for example) as follows:
development of serum-free systems;
nutrient optimization;
cell research;
cloning;
active substance research;
cell interactions;
organ simulation;
cytotoxicity testing;
cell processing;
cellular-based therapeutic formulation research;
automated implementation of complex cell cultures to reduce the necessity of animal experiments.

Further features of the invention read from the following description of preferred embodiments of the invention in conjunction with the drawings and the sub-claims, it being understood that each of the features may be achieved separately or in any combination thereof.

As evident from the drawings

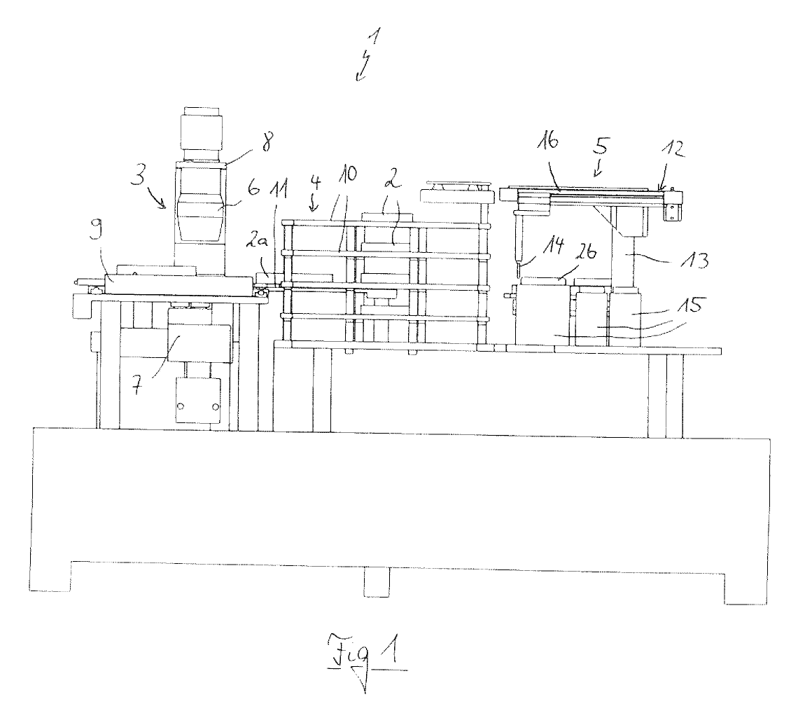

FIG. 1 is a side view of a device in accordance with the invention (without housing and climate chamber),

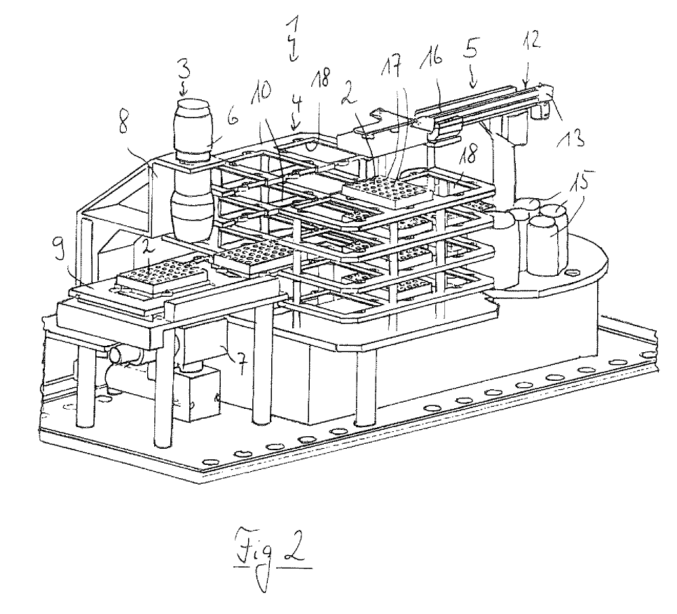

FIG. 2 is a view in perspective of the device as shown in FIG.

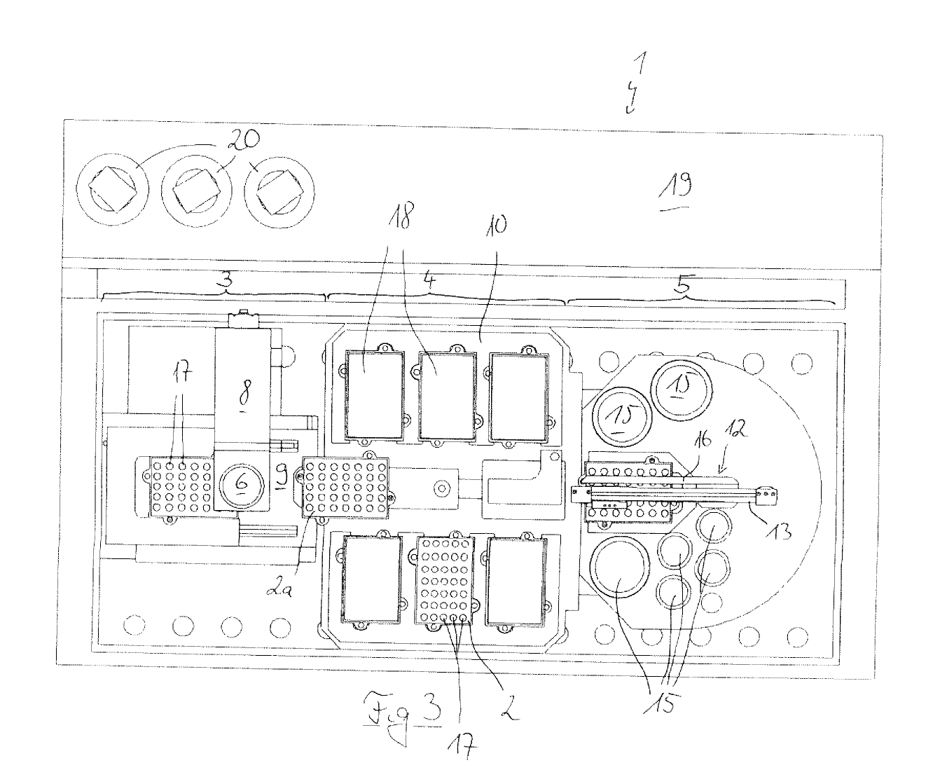

FIG. 3 is a top-down view of the device as shown in FIG. 1;

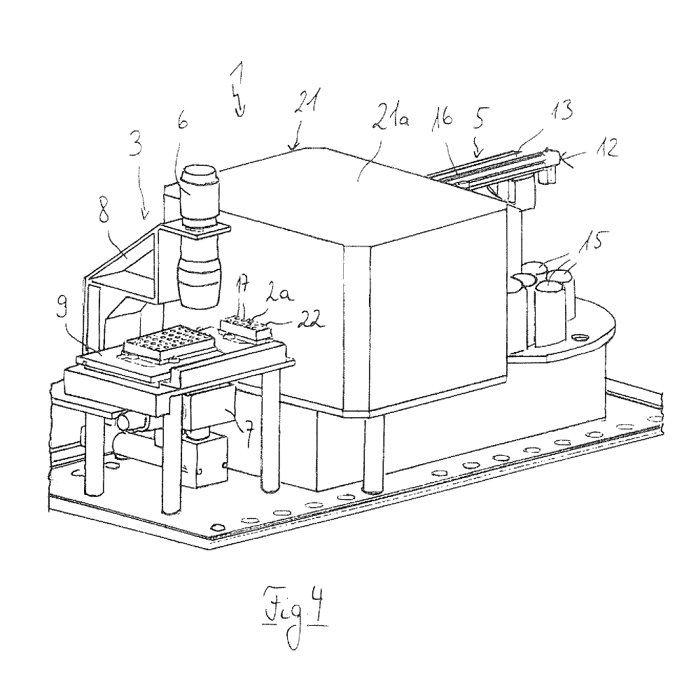

FIG. 4 is a view in perspective of the device as shown in FIG. 1 with climate chamber;

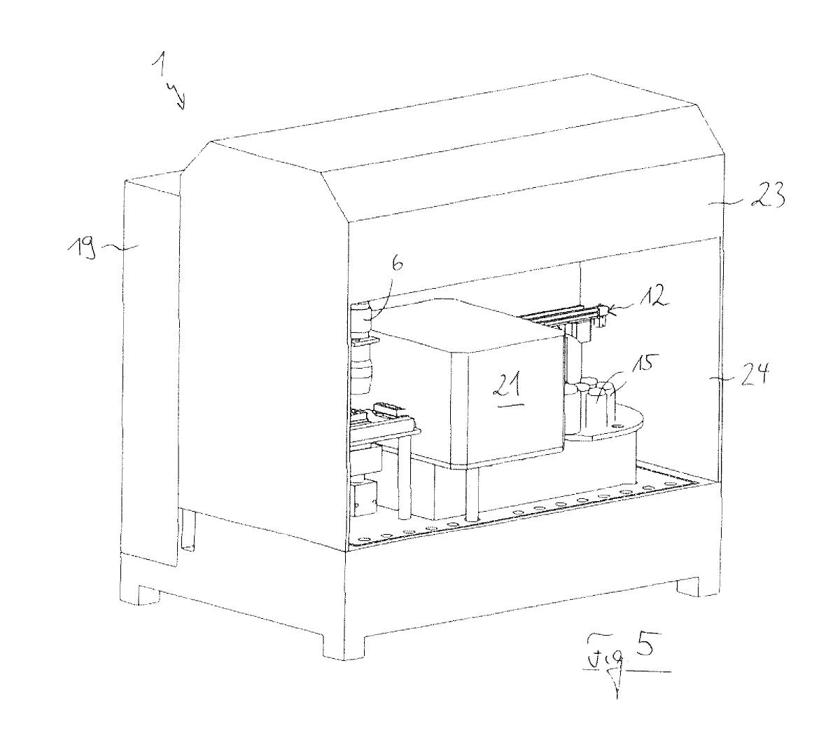

FIG. 5 is a view in perspective of the device as shown in FIG. 4 with housing;

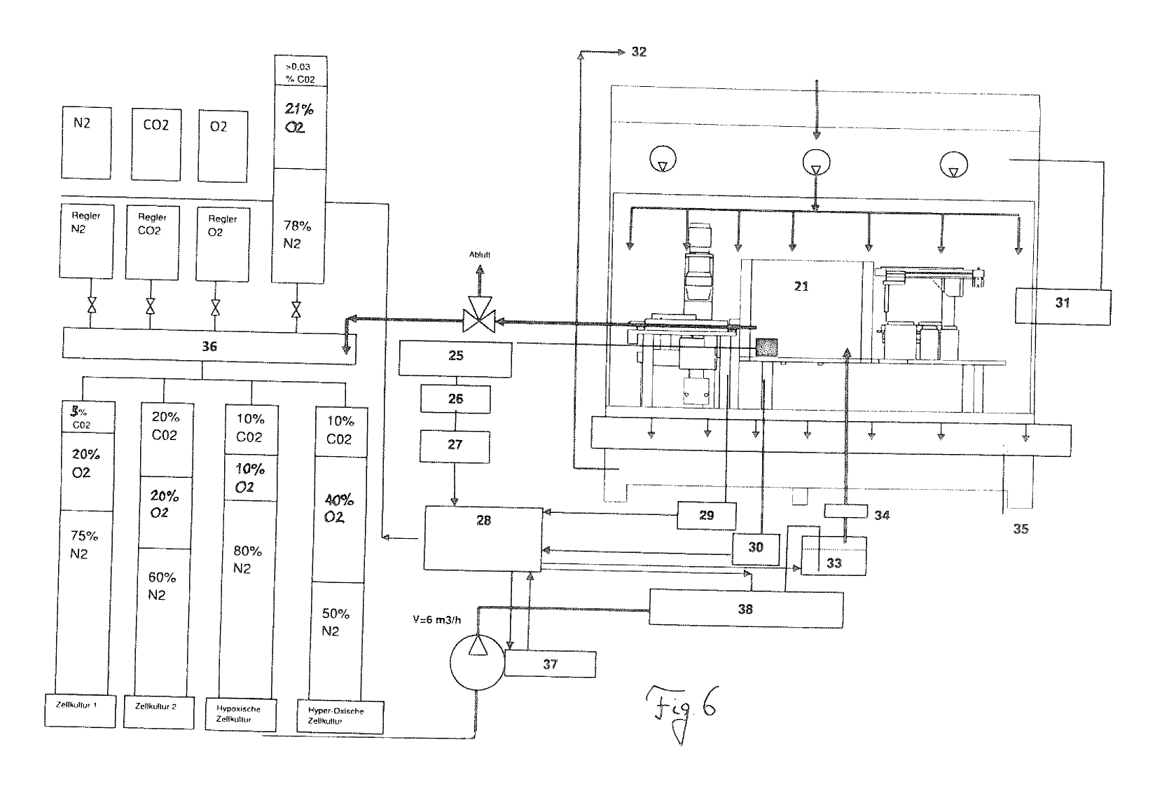

FIG. 6 is a diagram for open and closed loop control of a device in accordance with the invention;

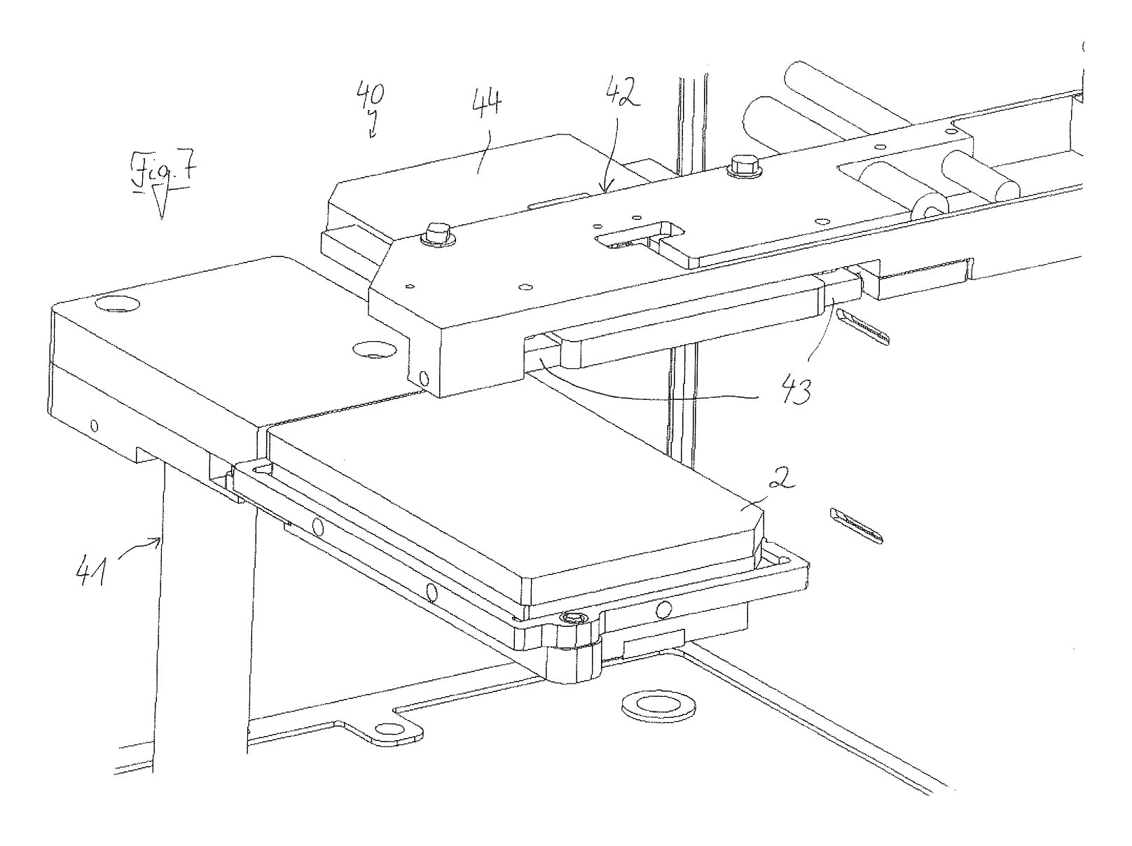

FIG. 7 is a view in perspective of an capper/uncapper in a device in accordance with the invention;

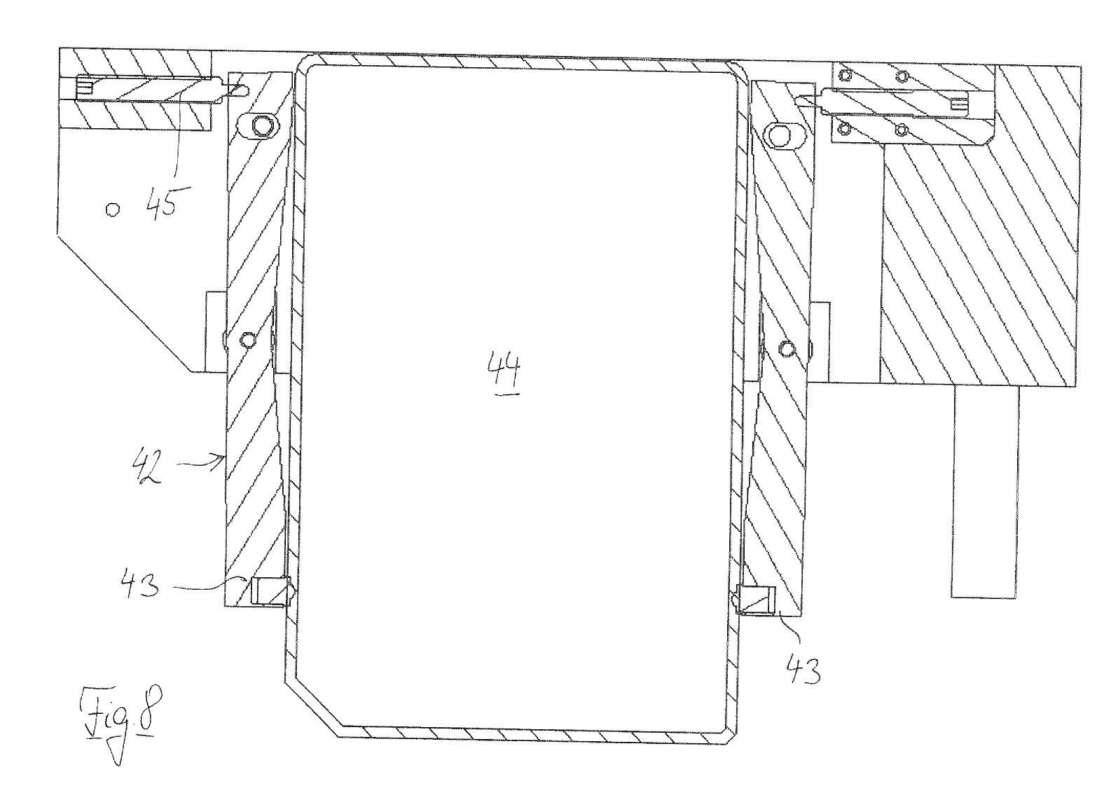

FIG. 8 is a top-down view of the capper/uncapper as shown in

FIG. 7.

Referring now to FIG. 1 there is illustrated a side view of a device 1 in accordance with the invention for automatically cultivating cells in parallel in microtiter (MT) plates 2 including an observation unit 3, a receptacle device 4 for receiving the MT plates 2 as well as a fluid distribution unit 5. The observation unit 3 comprises a CCD camera 6 as well as a microscope 7. The camera 6 as well as the microscope 7 are arranged on a support 8. The observation unit 3 comprises furthermore a receptacle table 9 to receive the MT plates 2.

The receptacle device 4 comprises a stack of four receptacle plates 10. The receptacle plates 10 comprise wells (not shown) for siting the MT plates 2. The receptacle device 4 can be raised and lowered. Within the receptacle plates 10 of the receptacle device 4 the MT plates 2 can be moved. Evident in this FIG. is how an MT plate 2a is transported from the receptacle device 4 into the region of the observation unit with the aid of a carriage 11. The up and down movements of the receptacle device 4 as well as the means for transporting the MT plates 2 within a plane make it possible to supply targeted MT plates 2, for example, to the observation unit or fluid distribution unit 5.

The fluid distribution unit 5 includes a microdispenser 12 with a moveable distributor arm 13 for executing particularly rotary and/or up and down movements, provided with a fixed or replaceable pipette 14. The fluid distribution unit 5 includes furthermore a plurality of receptacles 15 containing diverse fluids such as, for example, cell solutions, nutrients, stains, etc. The receptacles 15 are located in a defined arrangement about the microdispenser 12, the pipette 14 being positionable to the individual receptacles 15 by rotary and/or up and down movements of the distributor arm 13 and/or by movement of the pipette along the upper portion 16 of the distributor arm 13. To fill the wells 17 of the MT plates 2 the MT plates 2 can be transported automatically by the receptacle device 4 into the region of the fluid distribution unit 5 by means of a carriage. As shown in this FIG. an MT plate 2b is arranged in the region of the fluid distribution unit 5 where it is filled with specific fluids with the aid of the microdispenser 12,

Referring now to FIG. 2 there is illustrated a view in perspective of the device as shown in FIG. 1, clearly evident being the stacked arrangement of the receptacle plates 10 of the receptacle device 4, each of which comprises recesses 18 dimensioned so that the MT plates 2 can be received and held in place therein.

Referring now to FIG. 3 there is illustrated a top-down view of the device 1 as shown in FIG. 1 and FIG. 2. In addition to the actual working space located under the housing (not shown) a chamber 19 is arranged for receiving gas flasks 20.

Referring now to FIG. 4 there is illustrated the device 1 wherein the receptacle device 4 is arranged in a climate chamber 21. The housing 21a of the climate chamber 21 may be engineered transparent or non-transparent. The housing 21a closes off the receptacle device 4 substantially air-tight in thus helping to avoid contamination of the MT plates. Within the climate chamber 21 a desired claimate can be set by setting, for instance, the temperature, O₂ concentration, CO₂ concentration, N₂ concentration as well as the humidity in thus setting the cell cultures as required in each case for the cells being cultivated. The observation unit 3 and the fluid distribution unit 5 are arranged outside of the climate chamber 21 in thus not being exposed to the climatic parameters needed for optimum cell culture whilst preventing any contamination (e.g. lubricating grease) of the observation unit 3 or fluid distribution unit 5 from gaining access to the MT plates 2. Arranged in the housing 21a are two openings 22 permitting transportation of the MT plates 2 from/to the climate chamber 21. These openings 22 are arranged in the region of the observation unit 3 as well as in the region of the fluid distribution unit 5 where they permit transportation of MT plates 2, after which the openings 22 can be closed off.

Referring now to FIG. 5 there is illustrated the device 1 mounting the housing 23 which may be engineered completely transparent or non-transparent, it may also—as in the present example embodiment—comprise a transparent portion 24 in the form of a glass disc which may also be configured as an openable window so that, where necessary, access is provided through the open window 24 to the interior of the housing 23 The complete device 1 is configured as a so-called “sterile bench”. The housing 23 closes off the cultivation space substantially air-tight in preventing ingress of dirt, bacteria, etc from without into the inner space.

Each and every climatic condition in the interior of the climate chamber 21 or housing 23 is controlled by a computerized sensing and control system. Sensors in the interior of the housing 23 or in the interior of the climate chamber 21 continually furnish information as to each and every condition in each space, the sensing and control software maintaining the climatic conditions at a prescribed level. Also controlled by the computerized sensing and control system is the filling of the wells 17 of specific MT plates with specific amounts of specific fluids. For this purpose the MT plates 2 and the individual wells 17 of the MT plates 2 are indexed for recognition by the system. The computerized sensing and control system can be programmed so that specific wells 17 of selected MT plates 2 are filled at specific times with specific amounts of specific fluids.

Likewise, the computerized sensing and control system can be programmed for microscopic observation and evaluation of cell cultures in specific MT plates 2 particularly in specific wells of specific MT plates 2 at specific times by a specific technique, such as e.g. fluorescence, transmitted light or phase contrast microscopy and/or by any other methods of detection for observing and/or analyzing cells. Furthermore, the system can be programmed to image cell cultures at specific times, the images, as a rule, being saved for continuous evaluation.

Referring now to FIG. 6 there is illustrated a diagram for open and closed loop control of the climatic conditions in the interior of the climate chamber 21 or in the interior of the housing 23. Arranged in the climate chamber 21 are gas sensors 25, namely a CO₂ sensor 26, an N₂ sensor 27 as well as an O₂ sensor (not shown), it being understood that further sensors for other gases may be provided. The concentrations detected by the gas sensors are forwarded to a climate chamber closed-loop controller 28. A humidity sensor 29 continually detects the humidity in the interior of the climate chamber 21 and in the interior of the housing 23. Furthermore, a temperature sensor 30 continually senses the temperature. Both the humidity sensor 29 and the temperature sensor 30 forward their readings to the climate chamber closed-loop controller 28. A further temperature sensor 31 continually tracks the temperature in the interior of the housing 23 whilst a so-called laminar flow heater 32 sets a temperature in the housing preferably corresponding to the temperature in the climate chamber in thus preventing condensate forming in the region of the climate chamber. Via a humidifier 33 the climate chamber closed-loop controller 28 controls the humidity in the interior of the climate chamber 21. Disposed between the humidifier 33 and the climate chamber 21 is a filter element 34. In addition, fresh air 35 can be introduced into the interior of the housing 23 and climate chamber 21. Via the climate chamber closed-loop controller 28 the flow of the gases from the gas flasks 20 is controlled via gas flow controls, resulting in specific quantities of gas being introduced into a mixing chamber 36. Via a gas circulating pump 37, likewise controlled in a closed loop by the climate chamber closed-loop controller 28, the wanted gas mixture is directed into the climate chamber 21, the wanted temperature of the gas mixture being achieved by a conduit heater 38.

It is understood that a device in accordance with the invention may also be configured differently than as depicted in the FIGs. Thus, the device for receiving the MT plates may also be configured as a stack of disks each rotatable independently of the other. The individual elements of the receptacle device may also be configured like conveyor belts achieved by a linear arrangement of the MT plates and linear transport thereof.

It is just as feasible furthermore that the climate chamber is engineered transparent at least in the region of the observation unit for microscoping or observing through the wall of the climate chamber so that there is no need to move the MT plates out of the climate chamber. For this purpose the climate chamber may also feature a recess in which a MT plate can be nested for observation.

Irrespective of the embodiment the positions of the MT plates need to be defined the same every time they are observed; in other words, the observation positions need to be reattained every time with high accuracy in multipass operation. The accuracy of the system is dicated by the accuracy of the outer dimensions of the MT plates, diverse variants of which exist as to how they are engineered. The MT plates may be bottomed by a film in thus being optically defined, with, however, certain deviations in the Z direction. In another embodiment of the MT plates, they are engineered with a glass disk and optically defined, they then having hardly any deviation in the Z direction.

When the MT plates are repeatedly presented to the optics the cell positions in the MT plates need to be compensated with the dimensional deviations in the system, this too, being done by the computerized sensing system.

Referring now to FIG. 7 there is illustrated a view in perspective of a capper/uncapper 40 in a preferred embodiment of the device in accordance with the invention, the capper/uncapper 40 comprising a robotic arm 41 and a cap holder 42. A top-down view of the cap holder 42 is evident from FIG. 8. The capper/uncapper 40 serves to cap and uncap MT plates 2 which as a rule are stored in the climate chamber for incubating with the cap closed whilst in the fluid or optics station the MT plates are to be operated on without a cap. This is why the MT plates repeatedly need to be capped and uncapped as made possible by the capper/uncapper 40. When, for instance, an MT plate is to be transported from the incubator to the optics station the robotic arm 41 fetches the corresponding MT plate from the selection location in the climate chamber (“shelving location”), after which the robotic arm 41 travels under and towards the cap holder 42. The cap holder 42 comprises horizontally shiftable clips 43 which are moveable by means of a spring or a mechnically moveable pin 45. In lifting the MT plates 2 to the cap holder 42 the cap 44 of the MT plates 2 is ultimately inserted in the cap holder 42 and held in place by the clips 43. The robotic arm 41 then travels with the MT plate 2 downwards, the cap 44 remaining in the cap holder 42. The capper/uncapper 40 is positioned in the climate chamber 21.

The robotic arm 41 then transports the opened MT plate 2 out of the climate chamber into the observation unit (optics station). Whilst the MT plate remains in the observation unit for a certain time, the cap 44 continues to be held in the cap holder 42.

On completion of the actions at the optics station the robotic arm 41 transports the open MT plate back from the optics station into the climate chamber where the robotic arm 41 transports the open MT plate 2 upwards in the direction of the cap holder 42 to be in conclusion joined to the cap 44 in a precise fit. The clips 43 then release the cap 44 as a result of which the cap is restored to the MT plates 2 so that the robotic arm 41 can then lower the MT plate to the desired parking location.

1. A device for automatically cultivating cells in parallel in cell culture vessels having a housing comprising:
an observation unit comprising optics, wherein said optics further comprise at least one microscope and at least one camera,
a receptacle device to receive cell culture vessels, and
a fluid distribution unit to automatically dispense fluid into, and to remove fluid from, the cell culture vessels;
wherein climatic conditions in the device can be controlled in a closed loop at least in a region of the cell culture vessels, wherein a climate chamber is provided in the housing, wherein a desired temperature, a particular gas composition, or a combination thereof, can be adjusted automatically, and further wherein a receptacle device having the cell culture vessels is at least partially integrated into said housing,
wherein said housing further provides gas vessels to house diverse gases, and wherein a gas stream from the gas vessels is controllable and wherein defined quantities of gas from the gas vessels are directed into a gas mixing chamber, and gases are mixed in said gas mixing chamber to create a gas mixture; and the gas mixture is directed from said gas mixing chamber into the climate chamber.

2. The device of claim 1, wherein the diverse gases are selected from the group consisting essentially of O₂, CO₂, and N₂, or a combination thereof, and wherein the gas mixture is brought to a desired temperature prior to introduction into the climate chamber.

3. The device of claim 1, wherein the cell culture vessels are transportable from and into the climate chamber, the observation unit, and the fluid distribution unit automatically by a carriage or robotic arm.

4. The device of claim 1, wherein the observation unit, the fluid distribution unit, or both, are arranged outside of the climate chamber.

5. The device of claim 1, wherein the receptacle device comprises at least two receptacle units, wherein said receptacle units are arranged in a stacked configuration, and wherein said receptacle units comprise recesses for supporting the cell culture vessels.

6. The device of claim 1, wherein the optics of the observation unit are moveable, and the movements of the optics are controllable.

7. The device of claim 1, wherein a cell culture vessel located in the region of the observation unit is moveable, and wherein the movements of the cell culture vessel are controllable.

8. The device of claim 1, wherein the microscope comprises a transmitted light a phase contrast, or a fluorescent microscope, or a combination thereof.

9. The device of claim 1, wherein the fluid distribution unit includes a plurality of receptacles to contain fluids for cell culture, and a microdispenser to dispense defined and controlled amounts of fluid into the cell culture vessels and to remove defined and controlled amounts of fluid from the cell culture vessels, wherein the movements and actions of the microdispenser are controllable.

10. The device of claim 1, wherein the cell culture vessels are indexed.

11. The device of claim 1, wherein the cell culture vessels are configured transparent.

12. The device of claim 1, further comprising sensors to sense all relevant parameters, including temperature, CO₂ concentration, O₂ concentration, N₂ concentration, humidity or the supply of fluids into the cell culture vessels, or a combination thereof.

13. The device of claim 1, further comprising a computerized sensing and control system to automatically sense and control at least one of the following parameters or steps in operation, a combination of the following parameters or steps in operation or preferably all of the following parameters or steps in operation:
temperature, particularly in the climate chamber;
O₂ concentration in the gas mixture, particularly in the climate chamber;
CO₂ concentration in the gas mixture, particularly in the climate chamber;
N₂ concentration in the gas mixture, particularly in the climate chamber;
dispensing fluid into, and removing fluid from, selected cell culture vessels, with selected quantities of selected fluids at selected times;
microscopic observation and evaluation of cell cultures in selected cell culture vessels at selected times by a selected technique, wherein said technique is selected from the group consisting of fluorescence, transmitted light, and phase contrast microscopy, or a combination thereof.

14. The device of claim 1, further comprising a capper/uncapper to cap and uncap cell culture vessels, wherein said capper/uncapper is controlled automatically.

15. The device of claim 1, further comprising pipettes for disposable or multiple application of selective fluids in the fluid distribution unit.

16. A method for automatically cultivating cells in parallel in cell culture vessels located in a cell culture system, comprising the following steps in operation:
placing cell culture vessels into a receptacle device;
placing the selected fluids in a fluid distribution unit;
placing gas flasks, containing gases, including separate flasks for O₂, CO₂, and N₂ into a housing;
programming the system for automatically setting the parameters for at least one of the following: temperature, concentration of O₂ concentration of CO, or concentration of N₂;
programming the system for automatically dispensing fluid into, and removing fluid from, selected cell culture vessels, with selected quantities of selected fluids at selected times; and
programming the system for microscopic observation and evaluation of cell cultures in selected cell culture vessels at selected times by a selected technique, wherein said technique is selected from the group consisting essentially of fluorescence, transmitted light and phase contrast microscopy.

17. The device of claim 1, wherein the cell culture vessels are the wells of microtiter plates.

18. The device of claim 17, wherein the cell culture vessels are transportable from and into the climate chamber, the observation unit, and the fluid distribution unit automatically by a carriage or robotic arm;
wherein the observation unit, the fluid distribution unit, or both, are arranged outside of the climate chamber;
wherein the receptacle device comprises at least two receptacle units, wherein said receptacle units are arranged in a stacked configuration, and wherein said receptacle units comprise recesses for supporting the cell culture vessels;
wherein the optics of the observation unit are moveable, and the movements of the optics are controllable;
wherein a cell culture vessel located in the region of the observation unit is moveable, and wherein the movements of the cell culture vessel are controllable;
wherein the microscope comprises a transmitted light, a phase contrast, or a fluorescent microscope, or a combination thereof;
wherein the fluid distribution unit includes a plurality of receptacles to contain fluids for cell culture, and a microdispenser to dispense defined and controlled amounts of fluid into the cell culture vessels and to remove defined and controlled amounts of fluid from the cell culture vessels; and
wherein the movements and actions of the microdispenser are controllable.

19. The method of claim 16, wherein the cell culture vessels are the wells of microtiter plates.
